(19) United States
(12) Patent Application Publication
(10) Pub. No.: US 2007/0032824 A1
(43) Pub. Date: Feb. 8, 2007

(54) Tissue puncture closure device with track plug

(75) Inventor: Russell D. Terwey, St. Michael, MN (US)

Correspondence Address: L. Grant Foster;HOLLAND & HART LLP, 555 - 17th Street, Suite 3200, P.O. Box 8749, Denver, CO, 80201, US

(73) Assignee: St. Jude Medical Puerto Rico B.V.

(21) Appl. No.: 11/197,383

(22) Filed: Aug. 4, 2005

(51) Int. Cl. A61B 17/04

(52) U.S. Cl. 606/232

(57) ABSTRACT

Method and apparatus for sealing a puncture and/or incision formed percutaneously in a tissue are disclosed. The sealing may be facilitated by an anchor, a sealing plug and an incision track plug. A filament or other connector may attach the anchor to the sealing plug and the incision track plug. The incision track plug absorbs blood from surrounding tissue of an incision track. Some methods and apparatus provide for disengagable automatic tamping and/or cinching of the sealing plug and incision track plug when the apparatus is withdrawn from the puncture site.

### FIELD OF THE INVENTION

This invention relates generally to medical devices and more particularly to devices for sealing punctures or incisions in a tissue wall.

### BACKGROUND

Various surgical procedures are routinely carried out intravascularly or intraluminally. For example, in the treatment of vascular disease, such as arteriosclerosis, it is a common practice to invade the artery and insert an instrument (e.g., a balloon or other type of catheter) to carry out a procedure within the artery. Such procedures usually involve the percutaneous puncture of the artery so that an insertion sheath can be placed in the artery and thereafter instruments (e.g., a catheter) can pass through the sheath and to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral arterial punctures, is typically stopped by utilizing vascular closure devices, such as those described in U.S. Pat. Nos. 6,179,963; 6,090,130; and 6,045,569 and related patents, which are hereby incorporated by reference.

Typical closure devices such as the ones described in the above-mentioned patents place a sealing plug at the tissue puncture site. Nevertheless, the incision track leading to the invaded artery often continues to ooze blood from side vessels at the puncture site. Manual compression is typically applied at the puncture site to stop the track bleeding. Manual compression can lead to patient soreness and requires additional time from medical personnel. The time spent by medical personnel compressing the puncture site to stop the bleeding from the incision track can be expensive to the patient, and tiring to the medical personnel. Accordingly, there is a need for improving the sealing methods and apparatus at the site of subcutaneous tissue punctures.

### SUMMARY

The present invention addresses the above-described needs and others. Specifically, the present invention provides methods and systems for closing tissue punctures. However, unlike prior systems, the present invention reduces bleeding from incision tracks. Therefore, medical personnel may be able to spend less time closing tissue punctures and may be able to dispense with compression of the punctures. In addition, some embodiments provide automatic tamping to an incision track plug and a sealing plug as the closure device is retracted. Moreover, the present invention allows the automatic tamping system to disengage, facilitating full retraction of the closure device and easy separation of the incision track and sealing plugs from the remainder of the closure device.

In one of many possible embodiments, the present invention provides a tissue puncture closure device for partial insertion into and sealing of an internal tissue wall puncture. The device comprises a filament extending from a first portion of the closure device to a second portion of the closure device, an anchor for insertion through the internal tissue wall puncture attached to the filament at the second portion of the closure device, a sealing plug slidingly attached to the filament adjacent to the anchor, and an incision track plug slidingly attached to the filament adjacent to the sealing plug. The incision track plug may comprise a rigid biologically resorbable core with an adsorbent biologically resorbable outer lining. The incision track plug may also comprise a generally cylindrical hollow core covered with collagen. The incision track plug may comprise a generally cylindrical biologically resorbable polymer covered by a collagen pad. The incision track plug may be adapted to remain in an incision track and adsorb blood from surrounding skin tissue.

Some embodiments comprise a tamping tube slidingly disposed about the filament proximal of the incision track plug. The tamping tube may have an inner diameter, and the incision track plug may comprise a core having an outer diameter larger than the inner diameter of the tamping tube. When forced distally, the tamping tube may press the incision track plug toward the sealing plug, and the incision track plug may compress the sealing plug toward the anchor.

Some embodiments may further comprise a selectably disengagable automatic driving mechanism for automatically tamping or cinching the incision track plug and the sealing plug toward the second end upon withdrawal of the closure device from the internal tissue wall puncture. These embodiments may further comprise a tamping tube disposed adjacent to the incision track plug, and the tamping tube may be driven by a selectably disengagable automatic driving mechanism to force the incision track plug distally and tamp the sealing plug.

Some embodiments may further comprise a selectably disengagable automatic driving mechanism operatively connected to the incision track plug, the selectably disengagable automatic driving mechanism comprising a first gear and a spool assembly arranged on a first axis with a portion of the filament wound thereon, and a manually operated clutch between the first gear and the spool assembly. The clutch operably connects and disconnects the spool to the first gear. The embodiments may further comprise a second gear on a second axis adjacent to the first gear, and a third gear on a third axis adjacent to the second gear.

Some embodiments of the present invention provide a tissue puncture closure device for partial insertion into and sealing of a tissue puncture in an internal tissue wall accessible through a percutaneous incision track. The closure device may comprise an anchor for disposition on a distal side of the internal tissue wall, a sealing plug for disposition on a proximal side of the internal tissue wall, an incision track plug for disposition in the percutaneous incision track proximal of the sealing plug, a connector attached to and anchored at a distal end to the anchor, where the sealing plug and the incision track plug are slidably attached to the connector proximal of the anchor, and a tamping device is disposed on the connector for driving the incision track plug and the sealing plug along the connector distally towards the anchor. The incision track plug may comprise a stiff biologically resorbable core with an adsorbent biologically resorbable outer lining. The incision track plug may comprise a generally cylindrical hollow core covered with collagen, and the connector may be threaded through the cylindrical hollow core. The incision track plug may be adapted to compress the sealing plug toward the anchor, remain in an incision track, and adsorb blood from surrounding tissue. The incision track plug may comprise a biologically resorbable member approximately one inch long.

Another aspect of the present invention provides a method of sealing a tissue puncture in an internal tissue wall accessible through a percutaneous incision. The method comprises setting an anchor inside the internal tissue wall through the puncture, deploying a sealing plug and an incision track plug in the percutaneous incision, tamping the incision track plug and the sealing plug toward the anchor, and seating the sealing plug against the puncture. The method may further comprise adsorbing blood from surrounding tissue of the percutaneous incision with the incision track plug. The method may comprise leaving the anchor, sealing plug, and incision track plug in a patient body. Tamping may comprise manually tamping the incision track plug with a tamping tube, such that the incision track plug in turn tamps the sealing plug. According to some aspects, tamping comprises withdrawing a closure device carrying the sealing plug and the incision track plug from the tissue puncture, and automatically transducing a motive force generated by withdrawal of the closure device in a first direction to a cinching or tamping force in a second direction. The method may include manually disabling the tamping force in the second direction. Seating the sealing plug may comprise cinching the sealing plug and the anchor together across the puncture.

Another aspect of the invention provides a method of sealing a tissue puncture in an internal tissue wall accessible through a percutaneous incision. The method comprises providing a tissue puncture closure device comprising a filament connected at its distal end to an anchor, and to a sealing plug and incision track plug located proximal of the anchor, for disposition and anchoring about the tissue puncture. The method also includes inserting the tissue puncture closure device into the percutaneous incision, deploying the anchor into the tissue puncture, at least partially withdrawing the closure device from the percutaneous incision, and tamping the incision track plug and sealing plug toward the anchor upon withdrawal of the closure device from the internal tissue wall puncture. The tissue puncture closure device may comprise an automatic tamping device. The method may further comprise disengaging the automatic tamping device, retracting the tissue puncture closure device, exposing the filament, cutting the filament, and leaving the anchor and the sealing plug at the tissue puncture and the incision track plug in the percutaneous incision.

Additional advantages and novel features of the invention will be set forth in the description which follows or may be learned by those skilled in the art through reading these materials or practicing the invention. The advantages of the invention may be achieved through the means recited in the attached claims.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 is a partial cut-away side view of a tissue closure device according to the one embodiment of the present invention.

FIG. 2 is a side view of the tissue closure device of FIG. 1 engaged with an artery, which is shown in cross section, according to one embodiment of the present invention.

FIG. 3 is a side view of the tissue closure device of FIG. 1 being withdrawn from an artery to deploy a collagen sponge and a track plug.

FIG. 4 is a side view of the tissue closure device of FIG. 1 illustrating tamping of the collagen sponge and track plug according to one embodiment of the present invention.

FIG. 5 is a side view of the collagen sponge and track plug remaining at a puncture site following release from the tissue closure device according to one embodiment of the present invention.

FIG. 6 is an enlarged perspective view of the track plug according to one embodiment of the present invention.

FIG. 7A is a perspective assembly view of a tissue puncture closure device with an automatic tamping or driving mechanism and track plug according to one embodiment of the present invention.

FIG. 7B is a side view of the tissue closure device of FIG. 7A inserted into a procedure sheath and shown engaged with an artery in a first position according to one embodiment of the present invention.

FIG. 7C is a detailed inset of FIG. 7B.

FIG. 7D is a side view of the tissue closure device of FIG. 7A shown engaged with an artery in a second position retracting the procedure sheath according to one embodiment of the present invention.

FIG. 7E is a detailed inset of FIG. 7D.

FIG. 7F is a side view of the tissue closure device of FIG. 7A shown engaged with an artery in a third position tamping a sealing plug according to one embodiment of the present invention.

FIG. 7G is a detailed inset of FIG. 7F.

FIG. 8 illustrates the driving mechanism of FIG. 7A in a perspective assembly view with a carrier tube removed for clarity according to one embodiment of the present invention.

FIG. 9 is a side cross sectional view of the driving mechanism of FIG. 8 according to one embodiment of the present invention.

FIG. 10 is an enlarged perspective view of a portion of the driving mechanism and handle of FIG. 7A according to one embodiment of the present invention.

FIG. 11 is a perspective assembly view of a tissue puncture closure device with an automatic tamping or driving mechanism according to another embodiment of the present invention.

Throughout the drawings, identical reference numbers designate similar, but not necessarily identical, elements.

### DETAILED DESCRIPTION

As mentioned above, vascular procedures are conducted throughout the world and require access to an artery through a puncture. Most often, the artery is a femoral artery. To close the puncture following completion of the procedure, many times a closure device is used to sandwich the puncture between an anchor and a sealing plug. However, sometimes the incision track leading to the puncture bleeds for a prolonged period and must be compressed until the bleeding subsides. The present invention describes methods and apparatus that facilitate sealing punctures in arteries and other tissues and also reducing or containing bleeding from the incision tracks associated with the punctures. While the vascular instruments shown and described below include procedure sheaths and puncture sealing devices, the application of principles described herein are not limited to the specific devices shown. The principles described herein may be used with any medical device. Therefore, while the description below is directed primarily to arterial procedures and certain embodiments of a vascular closure device, the methods and apparatus are only limited by the appended claims.

As used in this specification and the appended claims, the term “tamp” or “tamping” is used broadly to mean packing or moving down by one or a succession of blows or taps or smooth, steady pressure, but not by excessive force. “Engage” and “engagable” are also used broadly to mean interlock, mesh, or contact between two devices. Likewise “disengage” or “disengagable” means to remove or capable of being removed from interlock, mesh, or contact. A “spool” is a cylinder or other device on which something else is at least partially wound. A “tube” is an elongated device with a passageway. The passageway may be enclosed or open (e.g. a trough). A “lumen” refers to any open space or cavity in a bodily organ, especially in a blood vessel. “Slidingly mounted” means movable relative to an appropriate support. A “detent” is a catch or lever that locks, at least temporarily, the movement of one part of a mechanism. “Free floating” means able to move freely according to at least one degree of freedom, at least after overcoming any initial holder. “Free floating” movement is not necessarily unlimited, and may include free movement only within a specified range. “Transduce” means to convert a force or other input energy in one form into output energy or forces of another form or direction. The term “effecting” means producing an outcome, achieving a result, or bringing about. The words “including” and “having,” as used in the specification, including the claims, have the same meaning as the word “comprising.”

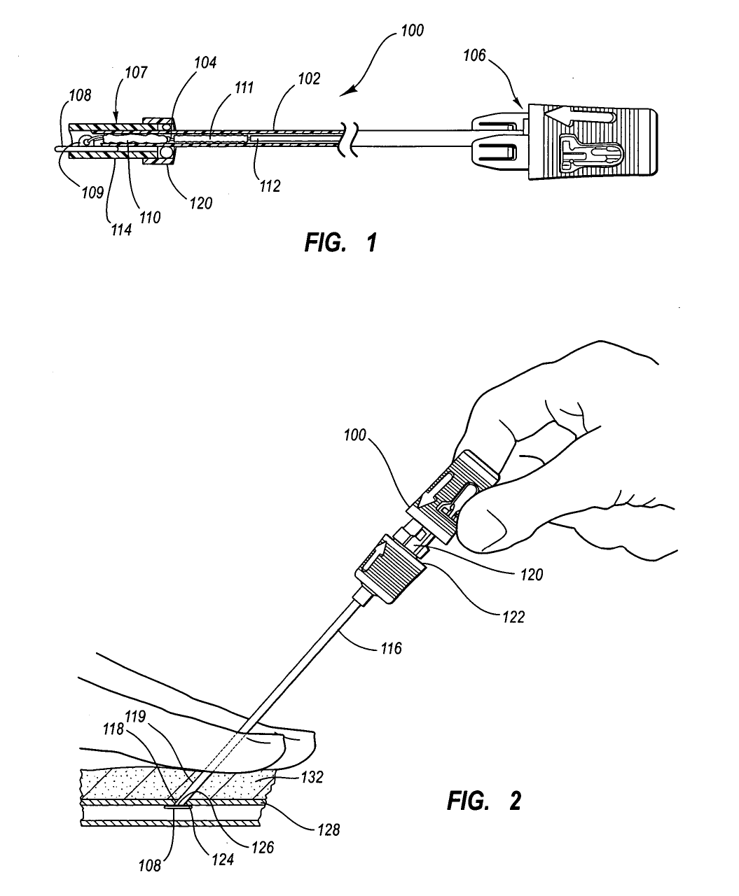

Referring now to the drawings, and in particular to FIGS. 1-4, an embodiment of a vascular puncture closure device 100 is shown in accordance with principles of the present invention. The vascular puncture closure device 100 includes a carrier tube 102 with a connector such as a filament or suture 104 extending at least partially therethrough. The closure device 100 also includes a first or proximal end 106 and a second or distal end 107. External to the second or distal end 107 of the carrier tube 102 is an anchor 108. The anchor is an elongated, stiff, low profile member including an eye 109 formed at the middle. The anchor 108 may be made of a biologically resorbable polymer.

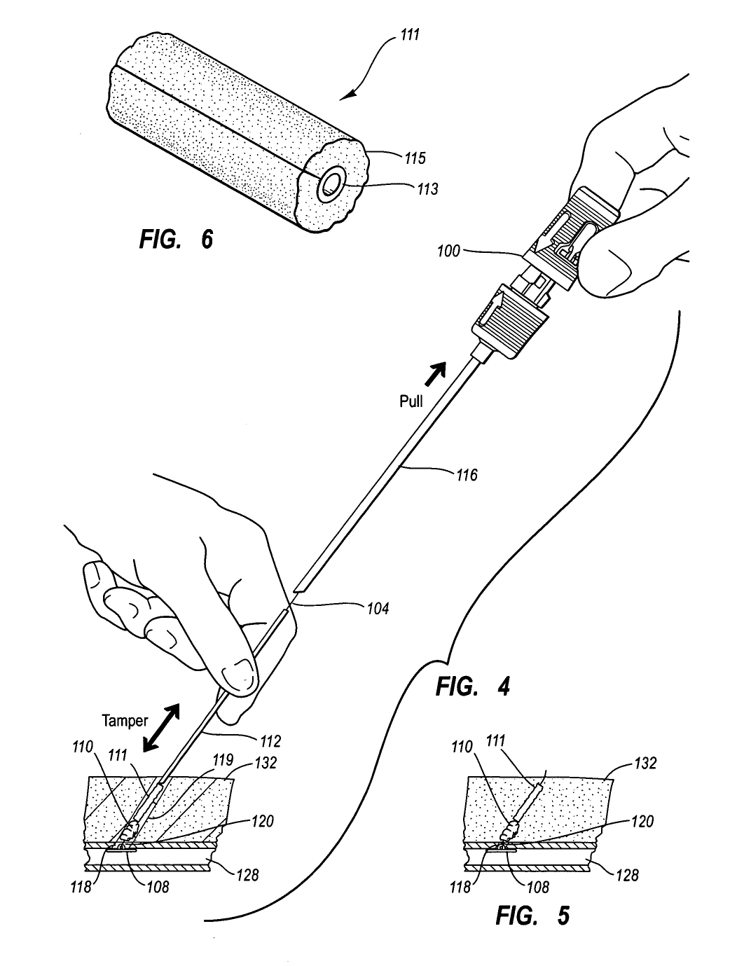

The suture 104 is threaded through the anchor 108 and back to a sealing plug such as collagen pad 110. The collagen pad 110 may be comprised of randomly oriented fibrous material bound together by chemical means. The collagen pad 110 is slidingly attached to the suture 104 as the suture passes distally through the carrier tube 102, but as the suture traverses the anchor 108 and reenters the carrier tube 102, it may be securely slip knotted proximal to the collagen pad 110 to facilitate cinching of the collagen pad 110 to the anchor 108 when the closure device 100 is properly placed and the anchor 108 is deployed (see FIG. 4).

The suture 104 is also threaded through an incision track plug 111 that is located proximal of and adjacent to the collagen pad 110. The incision track plug 111 may adsorb blood that may tend to ooze from incision tracks leading to internal tissue punctures. The incision track plug 111 is shown and described in detail below with reference to FIG. 6.

The carrier tube 102 may include a tamping tube 112 disposed therein. The tamping tube 112 is slidingly mounted on the suture 104 proximal of the incision track plug 111 and may be used by an operator to tamp the incision track plug 111, and the incision track plug 111 may in turn tamp the collagen pad 110 toward the anchor 108 at an appropriate time to seal a percutaneous tissue puncture.

Prior to deployment of the anchor 108 within an artery, the eye 109 of the anchor 108 rests outside the distal end 107 of the carrier tube 102. The anchor 108 may be temporarily held in place substantially flush with the carrier tube 102 by a bypass tube 114 disposed over the distal end 107 of the carrier tube 102.

The substantially flush arrangement of the anchor 108 and carrier tube 102 allows the anchor 108 to be inserted into a procedure sheath such as insertion sheath 116 as shown in FIGS. 2-4, and eventually through an arterial puncture 118. The insertion sheath 116 is shown in FIGS. 2-4 inserted through a percutaneous incision track 119 and into an artery 128. However, the bypass tube 114 (FIG. 1) includes an oversized head 120 that prevents the bypass tube 114 from passing through an internal passage of the insertion sheath 116. Therefore, as the puncture closure device 100 is inserted into the insertion sheath 116, the oversized head 120 bears against a surface 122 of the insertion sheath 116. Further insertion of the puncture closure device 100 results in relative sliding movement between the carrier tube 102 (FIG. 1) and the bypass tube 114, releasing the anchor 108 from the bypass tube 114 (FIG. 1). However, the anchor 108 remains in the substantially flush arrangement shown in FIG. 1 following release from the bypass tube 114, limited in movement by the insertion sheath 116.

As shown in FIG. 2, the insertion sheath 116 may include a monofold 124 at a second or distal end 126 thereof. The monofold 124 acts as a one-way valve to the anchor 108. The monofold 124 is a plastic deformation in a portion of the insertion sheath 116 that elastically flexes as the anchor 108 is pushed out through the distal end 126 of the insertion sheath 116. Typically, after the anchor 108 passes through the distal end 126 of the insertion sheath 116 and enters the artery 128, the anchor 108 is no longer constrained to the flush arrangement with respect to the carrier tube 102 and it deploys and rotates to the position shown in FIG. 2.

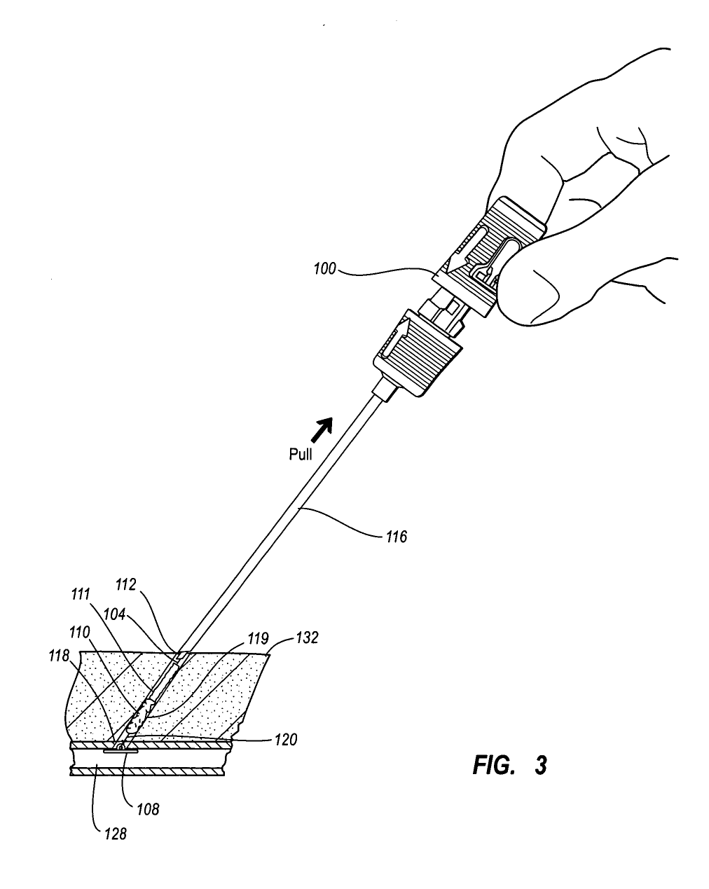

Referring next to FIGS. 3-4, with the anchor 108 deployed, the puncture closure device 100 and the insertion sheath 116 are withdrawn together, ejecting the collagen pad 110 and the incision track plug 111 from the carrier tube 102 and into the incision track 119 and exposing the tamping tube 112. With the tamping tube 112 fully exposed as shown in FIG. 4, the incision track plug 111 is manually tamped, the incision track plug 111 tamps the collagen pad 110, and the anchor 108 and collagen pad 110 are cinched together and held in place with the self-tightening slip-knot on the suture 102. Thus, the tissue puncture is sandwiched between the anchor 108 and the collagen pad 110, thereby sealing the tissue puncture 118. The incision track plug 111 remains percutaneously located in the incision track 119 and adsorbs blood from the surrounding skin 132 or other tissue. Because the incision track plug 111 adsorbs blood from the incision track 119, there is likely to be very little, if any, bleeding from the incision track. Accordingly, there may be little or no need for medical personnel to compress the puncture 118 and/or the incision track 119 to stop bleeding. The medical personnel may concentrate on other matters and the patient is likely to be less sore because of the reduction or elimination of compression pressure. The suture 104 may then be cut and the incision tract 119 may be closed. The suture 104, anchor 108, collagen pad 110, and incision track plug 111 are generally made of resorbable materials and therefore remain in place while the puncture 118 heals as shown in FIG. 5.

Referring next to FIG. 6, an enlarged view of one embodiment of the incision track plug 111 is shown. The incision track plug 111 may comprise a rigid biologically resorbable core with an adsorbent biologically resorbable outer lining. For example, the incision track plug 111 may include a stiff, generally cylindrical, hollow core 113. However, the hollow core 113 may comprise any convenient shape. The hollow core 113 facilitates threading of the suture 104 (FIG. 4) or other connector therethrough, and a sliding relationship along the suture 104 (FIG. 4). The biologically resorbable material may comprise a polymer. The hollow core 113 may be covered or surrounded by collagen or a second collagen pad 115. The second collagen pad 115 may be comprised of randomly oriented fibrous material bound together by chemical means. The second collagen pad 115 may also be generally cylindrical and adhered, stretched around, or otherwise connected to the outer surface of the hollow core 113. The hollow core 113 and the second collagen pad 115 may be approximately the same length and be coterminous as shown. However, some embodiments of the hollow core 113 and the second collagen pad 115 may comprise different lengths. According to some embodiments, each of the hollow core 113 and the collagen pad 115 are approximately one inch in length, although other lengths may also be used without limitation.

Using the typical tissue puncture closure device 100 described above, however, it may sometimes be difficult to eject and tamp of the collagen pad 110 and/or the incision track plug 111. The insertion sheath 116 resists deformation as the collagen pad 110 and the incision track plug 111 are ejected from the carrier tube 102, and tamping cannot commence until the sheath 116 has been removed so as to expose the tamping tube 112 for manual grasping. Under certain conditions, removal of the sheath 116 prior to tamping the collagen pad 110 and the incision track plug 111 could cause the collagen pad 110 to retract or displace proximally from the tissue puncture 118, creating an undesirable gap 120 between the collagen pad 110 and the puncture 118. The gap 120 may remain even after tamping as shown in FIG. 4, and could possibly result in only a partial seal and bleeding from the tissue puncture 118.

Therefore, the according to some aspects of the present invention, the collagen pad and the incision track plug 111 may be automatically tamped. The present specification describes a medical device such as a tissue puncture closure device that is capable of retracting a procedural sheath relative to a closure device, and exposing a distal end of the closure device prior to ejecting a sealing plug. The closure device may also automatically drive the incision track plug 111 and sealing plug toward a tissue puncture upon withdrawal of the tissue puncture closure device from the tissue puncture site. The mechanism for automatically driving the sealing plug may be selectably disengagable.

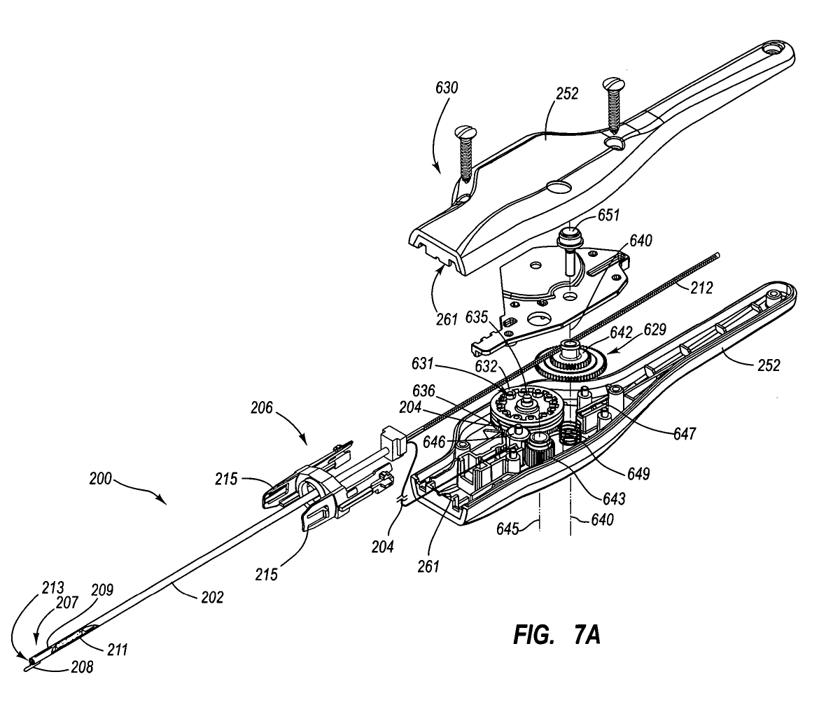

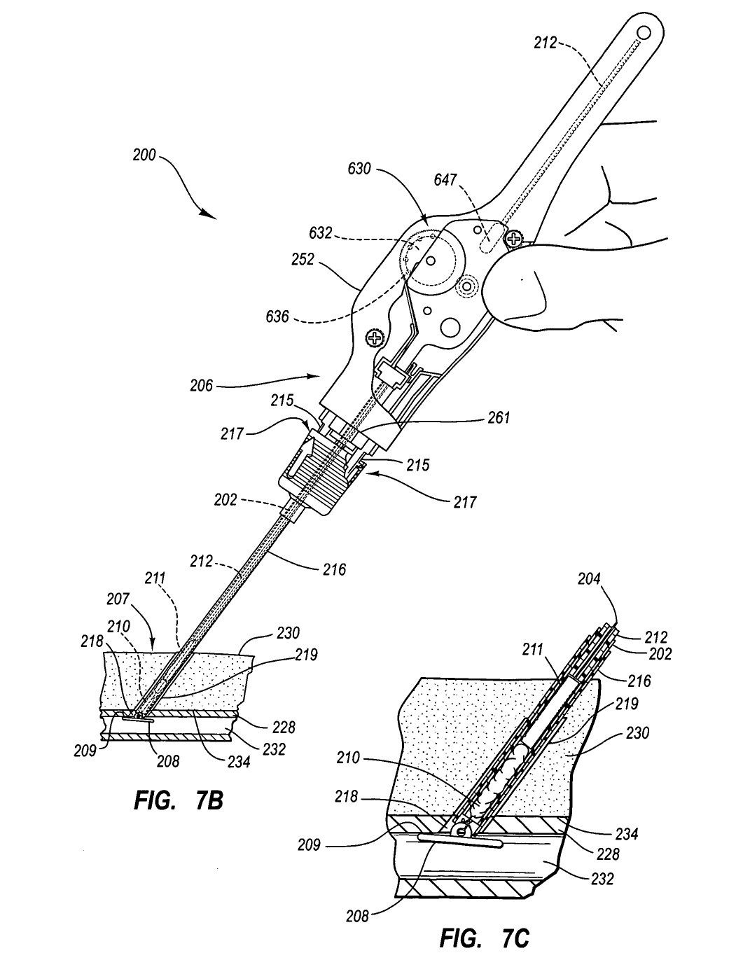

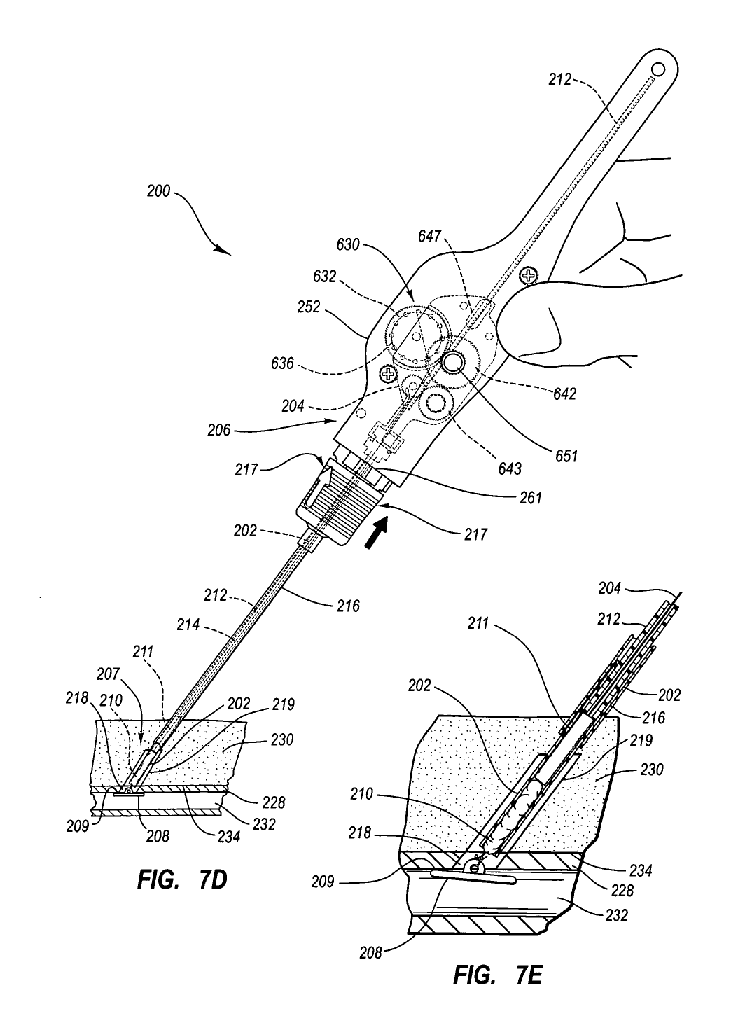

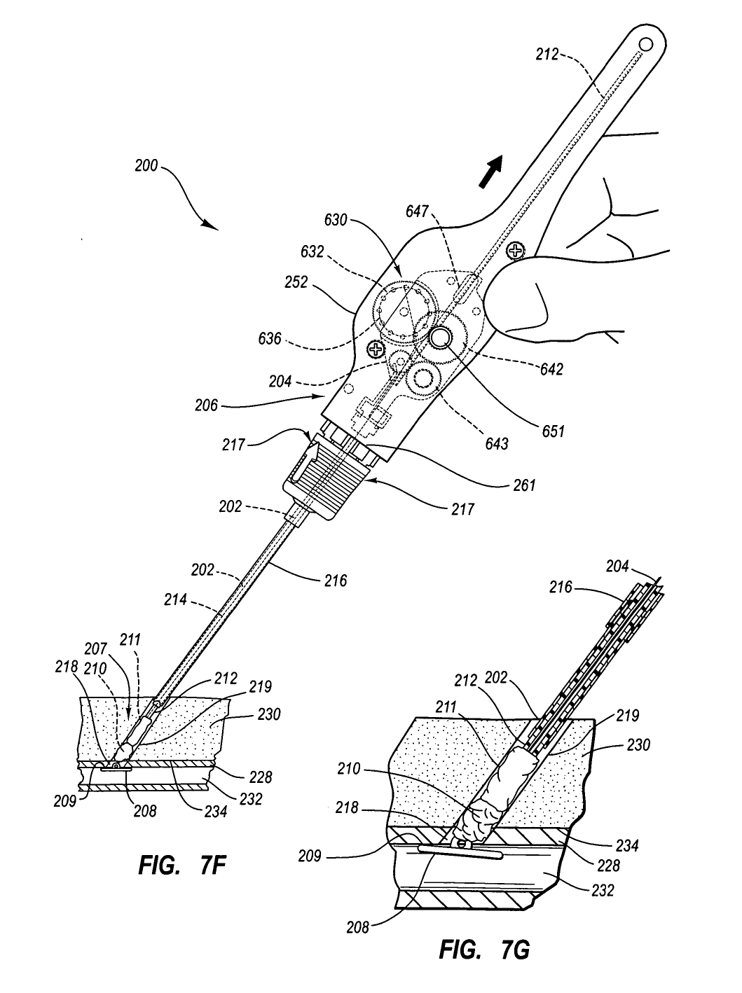

Referring next to FIGS. 7A-7G, a medical device, for example a tissue wall puncture closure device 200, is shown according to one embodiment of the present invention. The closure device 200 is shown in an assembly view in FIG. 7A. FIGS. 7B-7G illustrate the closure device 200 assembled and inserted through a procedure sheath 216 and into a lumen 232. The closure device 200 has particular utility when used in connection with intravascular procedures, such as angiographic dye injection, cardiac catheterization, balloon angioplasty and other types of recanalizing of atherosclerotic arteries, etc. as the closure device 200 is designed to cause immediate hemostasis of the blood vessel (e.g., arterial) puncture and from tissue surrounding the incision track. However, it will be understood that while the description of the preferred embodiments below are directed to the sealing off of percutaneous punctures in arteries and their associated incision tracks, such devices have much more wide-spread applications and can be used for sealing punctures or incisions in other types of tissue walls as well. Thus, the sealing of a percutaneous puncture in an artery, shown herein, is merely illustrative of one particular use of the closure device 200 of the present invention.

The closure device 200 includes a first or proximal end portion 206 and a second or distal end portion 207. A carrier tube 202 extends from the proximal end portion 206 to the distal end portion 207 and includes an outlet 213 at the distal end portion 207. The distal end portion 207 may include a slit 209.

The carrier tube 202 may be made of plastic or other material and is designed for insertion through the procedure sheath 216 (FIG. 7B). The procedure sheath 216 (FIG. 7B) is designed for insertion through a percutaneous incision 219 (FIG. 7B) in a tissue layer 230 and into the lumen 232. According to FIGS. 7B-7G, the lumen 232 comprises an interior portion of a femoral artery 228.

At the distal end portion 207 of the carrier tube 202 there is an anchor 208, a sealing plug 210, and an incision track plug 211 (FIG. 7B). The anchor 208 of the present embodiment is an elongated, stiff, low profile member arranged to be seated inside the artery 228 (FIG. 7B) against an artery wall 234 (FIG. 7B) contiguous with a puncture 218 (FIG. 7B). The anchor 208 is preferably made of a biologically resorbable polymer. The sealing plug 210 (FIG. 7B) is formed of a compressible sponge, foam, or fibrous mat made of a non-hemostatic biologically resorbable material such as collagen, and may be configured in any shape so as to facilitate sealing the tissue puncture 218 (FIG. 7B). The incision track plug 211 (FIG. 7B) is also made of biologically resorbable materials and may be similar or identical to the incision track plug 111 shown in FIG. 6.

The sealing plug 210, incision track plug 211, and anchor 208 are connected to one another by a connector such as a filament or suture 204 that is also biologically resorbable. The anchor 208, the sealing plug 210, the incision track plug 211, and the suture 204 are collectively referred to as the “closure elements” below. As shown in FIG. 7A, the anchor 208 is initially arranged adjacent to and exterior of the distal end portion 207 of the carrier tube 202, while the sealing plug 210 (FIG. 7B) and the incision track plug 211 are initially disposed within the carrier tube 202. The anchor 208 is shown nested in its low profile configuration along the carrier tube 202 to facilitate insertion into the lumen 232 in FIG. 7A, and deployed with a first surface 236 abutting the artery wall 234 in FIGS. 7B-7G. The suture 204 extends distally from the first end portion 206 of the closure device 200 through the carrier tube 202. The suture 204 may be threaded through the center of the incision track plug 211, through one or more perforations in the sealing plug 210, through a hole in the anchor 208, and proximally back toward the carrier tube 202 to the sealing plug 210. The suture 204 is preferably threaded again through a perforation or series of perforations in the sealing plug 210. The suture 204 may also be threaded around itself to form a self-tightening slip-knot. The suture 204 may thus connect the anchor 208 and the sealing plug 210 in a pulley-like arrangement to cinch the anchor 208 and the sealing plug 210 together when the carrier tube 202 is pulled away from the anchor 208, the sealing plug 210, and the incision track plug 211. The anchor 208 and the sealing plug 210 sandwich and lock the anchor and plug together, sealing the tissue puncture 218, and the incision track plug 211 sets in the incision 219 and adsorbs blood from the surrounding tissue.

The carrier tube 202 houses a tamping device, such as a tamping tube 212 (FIG. 7A), for advancing the incision track plug 211 and thus the sealing plug 210 along the suture 204 and toward the anchor 208. The tamping tube 212 is shown located partially within the carrier tube 202 and proximal of the sealing plug 208. The tamping tube 212, however, also extends through a handle 252 of the closure device 200. The tamping tube 212 is preferably an elongated tubular or semi-tubular rack that may be rigid or flexible and formed of any suitable material. For example, according to one embodiment, the tamping tube 212 is made of polyurethane. The suture 204 extends through at least a portion of the tamping tube 212. For example, as shown in FIGS. 7A-7G, the suture 204 extends along the tamping tube 212 between the first and second end portions 206, 207. However, the suture 204 is not directly connected to the tamping tube 212. Accordingly, the suture 204 and the tamping tube 212 may slide past one another.

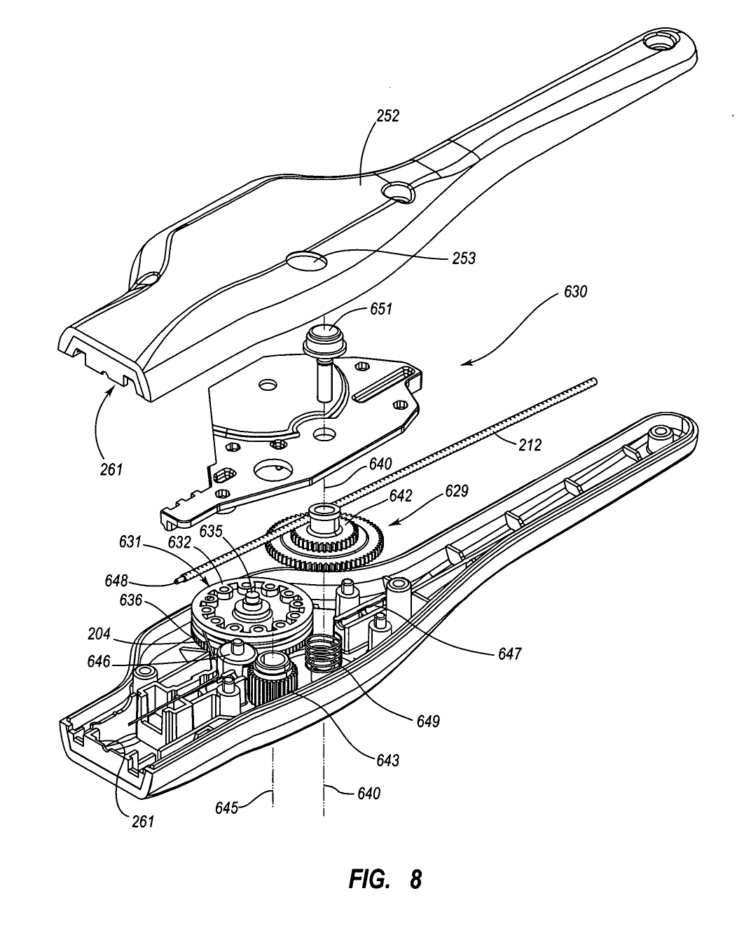

According to the embodiment of FIGS. 7A-7G, the suture 204 attaches to an automatic tamping assembly. The automatic tamping assembly may include an automatic driving mechanism 630 or other transducer, and the tamping tube 212. The automatic driving mechanism 630 is located within the housing or handle 252 at the first end portion 206 of the closure device 200. Embodiments of the automatic driving mechanism 630 are described in detail below with reference to FIGS. 8-11. The tamping tube 212 may comprise a rack receptive of gear teeth (discussed in more detail below).

In practice, the carrier tube 202 of the closure device 200 (containing the closure elements described above) is inserted into the insertion sheath 216, which is already inserted within the artery 228 (FIGS. 7B-7C). As the closure device 200 and the associated closure elements are inserted into the procedure sheath 216, the anchor 208 passes through and out of the distal end of the procedure sheath 216 and is inserted into the artery lumen 232. As mentioned above and shown in FIG. 7A, the anchor 208 is initially arranged substantially flush with the carrier tube 202 to facilitate insertion of the anchor 208 through the percutaneous incision 219 and into the lumen 232.

After the anchor 208 passes out of the distal end of the procedure sheath 216, however, it tends to deploy or rotate to the position shown in FIGS. 7B-7C. The closure device 200 may also be partially withdrawn from the insertion sheath 216, catching the anchor 208 on the distal end of the insertion sheath 216 and rotating it to the position shown in FIGS. 7B-7C. However, the closure device 200 preferably includes a pair of biased fingers 215 that are lockingly received by a matching pair of recesses 217 in the procedure sheath 216. The locking arrangement between the biased fingers 215 and matching recesses 217 preferably fixes the position of the handle 252 relative to the procedure sheath 216.

Following deployment of the anchor 208, the handle 252 and the insertion sheath 216 are withdrawn together. Withdrawing the handle 252 causes the anchor 208 to anchor itself within the artery 228 against the artery wall 234. With the anchor 208 anchored within the artery 228 at the puncture site 218, further retraction of the handle 252 and insertion sheath 216 tends to pull the incision track plug 211 and the sealing plug 210 out from the distal end portion 207 of the carrier tube 202, thereby depositing the incision track plug 211 and the sealing plug 210 within the incision or puncture track 219. The slit 209 (FIG. 7A) in the carrier tube 202 allows the distal end portion 207 of the carrier tube to flex or open, facilitating ejection of the incision track plug 211 and the sealing plug 210. However, the slit 209 (FIG. 7A) at the distal end portion 207 of the carrier tube 202 may be prevented from opening or flexing by the procedure sheath 216, which is concentric with the carrier tube 202. Therefore, according to some embodiments of the present invention, retraction of the handle 252 and insertion sheath 216 causes the insertion sheath 216 to retract with respect to the carrier tube 202 to a second position shown in FIGS. 7D-7E.

Referring to FIGS. 7D-7E, the distal end portion 207 of the carrier tube 202 is exposed (within the incision track 219) as the handle 252 and the procedure sheath 216 are retracted. The carrier tube 202 retains its position relative to the puncture 218 until the handle 252 and the procedure sheath 216 have been retracted a predetermined distance. Relative movement between the handle 252 procedure sheath 216 and the carrier tube 202 is facilitated by a sliding mount arrangement between the automatic driving mechanism 630 and the handle 252. However, according to some embodiments, the automatic driving mechanism 630 is fixed to the handle 252.

As shown by the combination of FIGS. 7B-7G, the automatic driving mechanism 630 (which is attached to the carrier tube 202) is preferably free floating or displaceable and slides relative to the handle 252 as the handle 252 and the procedure sheath 216 are retracted. However, the automatic driving mechanism 630 may be initially held in a first position relative to the handle 252 as shown in FIGS. 7B and 10. For example, as shown in FIG. 10, the automatic driving mechanism 630 may comprise a temporary holder such as a stowage detent 255 slidingly mounted in a track. The track is shown in FIG. 10 as a webbing track 253. The webbing track 253 is disposed in the handle 252. The webbing track 253 may have a first width W1 and a second width W2. The stowage detent 255 may include a finger 257 with a protrusion 259 biased to a third width W3 greater than the first width W1, but less than the second width W2. The finger 257 extends at least partially into the webbing track 253 at the second width W2 to at least temporarily hold the automatic driving mechanism 630 in the first position shown in FIGS. 7B and 10, and prevent premature sliding within the handle 252.

Although the finger 257 tends to hold or temporarily lock the automatic driving mechanism 630 in the first position shown in FIGS. 7B and 10, the finger 257 releases when a sufficient predetermined force is applied between the handle 252 and the automatic driving mechanism 630. For example, with the anchor 208 deployed, a retraction force provided by a user to the handle 252 causes the finger 257 to deflect inward and slide distally toward the first width W1 portion of the webbing track 253. When the protrusion 259 of the finger enters the first width W1, the stowage detent 255 is “released” and provides very little resistance to sliding movement between the automatic driving mechanism 630 and the handle 252. Accordingly, retraction of the handle 252 retracts the procedure sheath 216 (which is fixedly connected to the handle 252), but the automatic driving mechanism 630 and the carrier tube 202 slide relative to the handle 252 and therefore remain in position with respect to the puncture 218. The automatic driving mechanism 630 may slide a predetermined distance with respect to the handle 252 until the automatic driving mechanism 630 reaches a stop 261 (FIG. 7D). The predetermined distance is preferably at least long enough to fully expose the slit 209 (FIG. 7A) in the carrier tube 202.

When the automatic driving mechanism 630 reaches the stop 261 (FIG. 7D), further retraction of the handle 252 withdraws the carrier tube 202 as well, ejecting and tamping the incision track plug 211 and the sealing plug 210 automatically as shown in FIGS. 7F-7G. Therefore, some embodiments of the closure device 200 of the present invention automatically tamp the incision track plug 211 and the sealing plug 210. The incision track plug 211 and the sealing plug 210 are tamped while the carrier tube 202 is being withdrawn, reducing or eliminating any gaps that may otherwise occur between the sealing plug 210 and the puncture 218 in the femoral artery 228.

In addition, by placing tension on or pulling the suture 204 away from the puncture tract 219, the suture 204 may cinch and lock (with a slip knot or the like) together the anchor 208 and the sealing plug 210, sandwiching the artery wall 234 between the anchor 208 and sealing plug 210. The force exerted by the tamping tube 212 via the incision track plug 211 and the cinching together of the anchor 208 and sealing plug 210 by the filament 204 also causes the sealing plug 210 to deform radially outward within the puncture tract 219 and function as an anchor on the proximal side of the tissue puncture site 218 as shown in FIGS. 7F-7G.

The tamping tube 212 is automatically driven toward the sealing plug 210 by the automatic driving mechanism 630. One embodiment of the automatic driving mechanism 630 is shown in detail in FIG. 8. The automatic driving mechanism 630 may comprise a gearbox assembly 629, and the gearbox assembly 629 may be selectably disengagable. According to the embodiment of FIG. 8, once the automatic driving assembly 630 contacts the stop 261, further retraction of the closure device 200 automatically effects tamping of the incision track plug 211 and thus the sealing plug 210 (FIG. 7F).

According to the gearbox assembly 629 of FIG. 8, the suture 204 is connected to and partially wound about a spool 632 of a first gear and spool assembly 631. The first gear and spool assembly 631 includes both the spool 632 and a first gear 636 arranged on a first axis 635. According to the embodiment of FIG. 8, the first gear 636 is connected to the spool 632 and therefore they rotate together. Withdrawal of the closure device 200 (FIG. 7F) from the tissue puncture site 218 (if the anchor 208 (FIG. 7F) is deployed and the gearbox assembly 629 has contacted the stop 261) causes the suture 204 to unwind from the spool 632. The spool 632 rotates as the suture 204 unwinds and provides a torsional motive force that is transduced to a linear tamping force.

The torsional motive force provided by the spool 632 is transduced into the linear tamping force by the gearbox assembly 629 according to the embodiment of FIG. 8. The gearbox assembly 629 includes the first gear 636 arranged coaxially with the spool 632. As shown in FIG. 8, the first gear 636 may be arranged adjacent to a second gear 642. The second gear 642, when assembled, engages the first gear 636. The second gear 642 is arranged on a second axis 640. The second gear 642 may be a two-stage gear, with each stage engaging a different adjacent gear as shown. The first and second gears 636 and 642 may engage one another with a frictional fit, or with meshed gear teeth as shown. The second gear 642 is arranged adjacent to a third gear 643 on a third axis 645. When assembled, the second gear 642 engages and drives the third gear 643.

The tamping tube 212 is disposed between the third gear 643 and a guide 646. The tamping tube 212 preferably includes the teeth shown, which mesh with teeth of the third gear 643. A concave holder 647 may support the tamping tube 212. When the spool 632 rotates, it drives the tamping tube 212, which in turn tamps the incision track plug 211 and the sealing plug 210 (FIG. 7F). Alternatively, the tamping tube 212 may not extend into the housing 252, and instead a separate rack may mesh with the third gear 643. The separate rack would, in turn, drive the tamping tube 212.

The tamping tube 212 is preferably semi-tubular and partially disposed about the suture 204 along its longitudinal axis. The semi-tubular shape of the tamping tube 212 has a generally U-shaped cross section, and provides an open channel or trough 648 through which the suture 204 may enter and exit. The open channel 648 permits the suture and the tamping tube 212 to merge as the spool 632 unwinds. The suture 204 and the tamping tube 212 are not fixedly connected to one another, allowing each to slide freely past the other. Accordingly, with the anchor 208 (FIG. 7D) deployed, as the closure device 200 (FIG. 7F) is retracted in a first direction with the gearbox assembly 629 bearing against the stop 261 (FIG. 7F), the suture 204 unwinds from the spool 632, which drives the gearbox assembly 629. The gearbox assembly 629 drives the tamping tube 212 in a second, opposite direction, and the tamping tube tamps the incision track plug 111 and the sealing plug 210 (FIG. 7F).

It may be desirable in some cases to increase the linear velocity of the tamping tube 212 relative to the linear velocity at which the closure device 200 (FIG. 7F) is withdrawn. Increasing the linear velocity for the tamping tube 212 may better assure that the sealing plug 210 (FIG. 7F) is forced toward the anchor 208 (FIG. 7F) when the closure device 200 (FIG. 7F) is withdrawn in an opposite direction. Therefore, according to some embodiments, the gearbox assembly 629 may have an overall gear ratio greater than 1:1. For example, the gear ratio may range between approximately 1.5:1 and 3.0:1 for some embodiments, while the gear ratio is about 2.1:1 in other embodiments

However, it should be noted that the linear velocity of the tamping tube 212 should not be excessively greater than the linear velocity of withdrawal of the closure device, as excessive speed could potentially force the sealing plug 210 (FIG. 7F) through the tissue puncture 218 (FIG. 7F) and into the lumen 232 (FIG. 7F) of the artery 228 (FIG. 7F). Likewise, an insufficient opposing force against the anchor 208 (FIG. 7F) could potentially result in the anchor 208 (FIG. 7F) being pulled out of place from within the artery 228 (FIG. 7F). Therefore, according to some uses, the withdrawal force should not exceed approximately 2.5 pounds.

It will be understood by those of skill in the art having the benefit of this disclosure that the gearbox assembly 629 configuration shown in FIG. 8 is exemplary in nature, and not limiting. Any gear configuration (including a single gear) may be used to transmit a motive force generated by retraction of the suture 204 from the closure device 200 (FIG. 7F) to provide an automatic driving force to the sealing plug 210 (FIG. 7F) via the incision track plug 211 and the tamping tube 212.

As mentioned above, the gearbox assembly 629 may be selectable disengagable. Therefore, one or more of the spool 632, first gear 636, second gear 642, and third gear 643 may be movable to disengage or manually disable adjacent gears. For example, one or more of the first gear 636, second gear 642, or third gear 643 may be movable along its respective axis to disengage from an adjacent gear. As shown in FIG. 8, a biasing member such as a spring 649 is disposed at the second axis 640 biasing the second gear 642 into a meshed relationship with the first and third gears 636, 643. However, the second gear 642 is movable along the second axis 640 by operation of an actuator 651 coupled to the second gear 642. Therefore, a force may be applied to the actuator 651 (following sliding movement of the gearbox assembly 629 to reach the stop 261, thereby aligning the actuator 651 with an access hole 253 in the handle 252) laterally with respect to the second gear 642, to overcome a biasing force provided by the spring 649 and move or displace the second gear 642 axially out of the meshed or contacting relationship with at least one of the first and third gears 636, 643. According to the embodiment of FIG. 8, axial movement of the second gear 642 only disengages the second gear 642 from the first gear 636. Disengaging the gearbox assembly 629 allows retraction of the closure device 200 (FIG. 7F) and unwinding of the suture 204 from the spool 632 without driving the tamping tube 212. The advantages of this disengagement are discussed below with reference to the operation of the closure device 200.

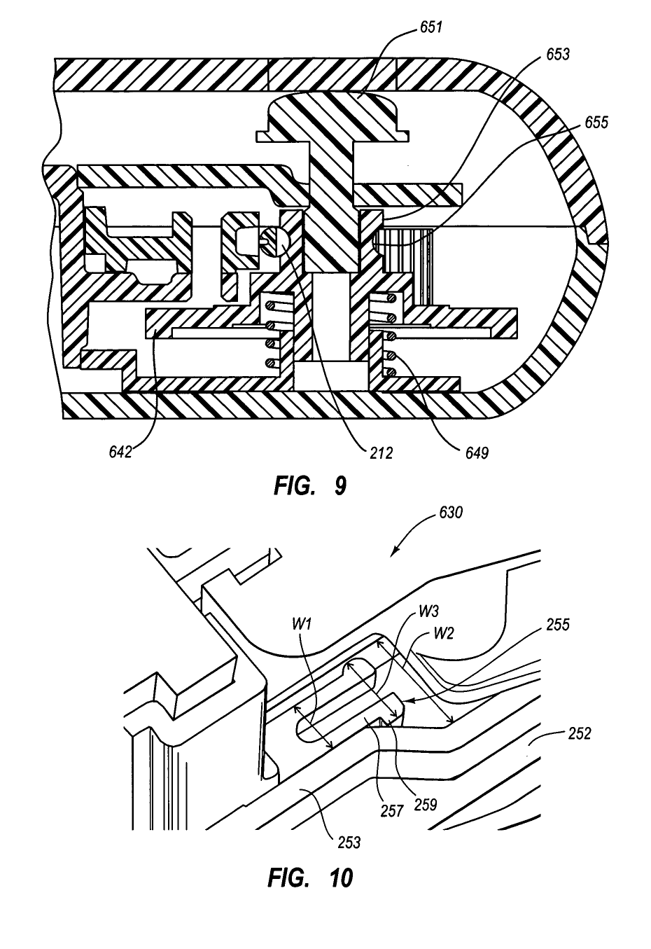

However, as shown in FIGS. 8-9, the tamping tube 212 may interlock with the second gear 642 in a first rack position shown, preventing premature activation of the actuator 651. The interlocking geometry is seen more clearly in FIG. 9. The second gear 642 may include a second gear hub 653 with an annular groove 655. The tamping tube 212 is disposed in the annular groove 655 in the first rack position, which locks out the actuator 651. The tamping tube rests on the concave holder 647. Therefore, as long as the tamping tube 212 is disposed in the annular groove 655, the actuator 651 may not be depressed. With the tamping tube 212 disposed in the annular groove 655, forces applied to the actuator 651 are transmitted to the second gear 642, but the second gear is prevented from moving axially by the rack disposed in the annular groove 655 and supported by the concave holder 647. Nevertheless, retracting the closure device 200 (FIG. 7F) results in rotation of the gears of the gearbox assembly 629, and linear movement of the tamping tube 212. When the tamping tube 212 has moved a predetermined distance to a second tamping tube position sufficient to cause effective tamping of the incision track plug 211 and the sealing plug 210 (FIG. 7F), the tamping tube 212 also moves out of the annular groove 655 (See FIG. 7F). Therefore, the actuator 651 is no longer locked out, and the second gear 642 may be disengaged once the tamping tube 212 has moved linearly the predetermined distance.

Operation of the embodiment of FIGS. 7A-10 may be as follows. As the handle 252 of the closing device 200 is retracted from the puncture tract 219 as shown in FIG. 7B, the detent 255 releases. The automatic tamping mechanism 630 and carrier tube 202 remain stationary and therefore float relative to the handle 252. The procedure sheath 216 is retracted as the handle 252 is withdrawn, exposing the distal end 207 of the carrier tube 202. The automatic tamping mechanism 630 eventually contacts a stop 261, and further retraction causes the automatic tamping mechanism 630 and carrier tube 202 to retract as well. As the automatic tamping mechanism 630 retracts, the suture 204, which is threaded through the anchor 208, unwinds from and causes rotation of the spool 632. The spool 632 drives the first gear 636 as it rotates via the coaxial connection between the spool 632 and the first gear 636. As the first gear 636 rotates, it drives the second gear 642. The second gear 642 drives the third gear 643, and the third gear 643 drives the tamping tube 212. The tamping tube 212 tamps the incision track plug 211 and the incision track plug 211 tamps the sealing plug 210. Therefore, as the closing device 200 is retracted from the puncture track 219, the procedure sheath 216 is retracted (FIGS. 7D-7E), the sealing plug 210 is automatically tamped (FIGS. 7F-7G), and the incision track plug 211 is strategically placed in the puncture track 219. The sealing plug 210 is more likely to create a sufficient arterial seal without a gap relative to the anchor 208, as may otherwise occur with a separate manual tamping procedure.

Moreover, when the sealing plug 210 has been sufficiently tamped, the selectably disengagable gearbox assembly 629 may be disengaged, enabling further retraction of the closure device 200 without additional tamping. With the sealing plug 210 fully tamped, there may be little or no portion of the suture 204 extending outside of the tissue layer 230 and exposed to an operator. Therefore, it may be difficult for an operator to separate the sealing plug 210, incision track plug 211, and anchor 208 from the remainder of the closure device 200. In addition, too much retraction with the selectably disengagable gearbox assembly 629 enabled could potentially overtamp the sealing plug 210 into the artery 228. Accordingly, the selectably disengagable gearbox assembly 629 may be advantageously disabled by activating the actuator 651 through the access hole 253. Activating the actuator 651 allows the suture 204 to fully unwind from the spool 632 without driving the tamping tube 212. Unwinding the spool 632 exposes a sufficient length of the suture 204 to allow an operator to easily cut it and separate the sealing plug 210, incision track plug 211, and anchor 208 from the remainder of the closure device 200.

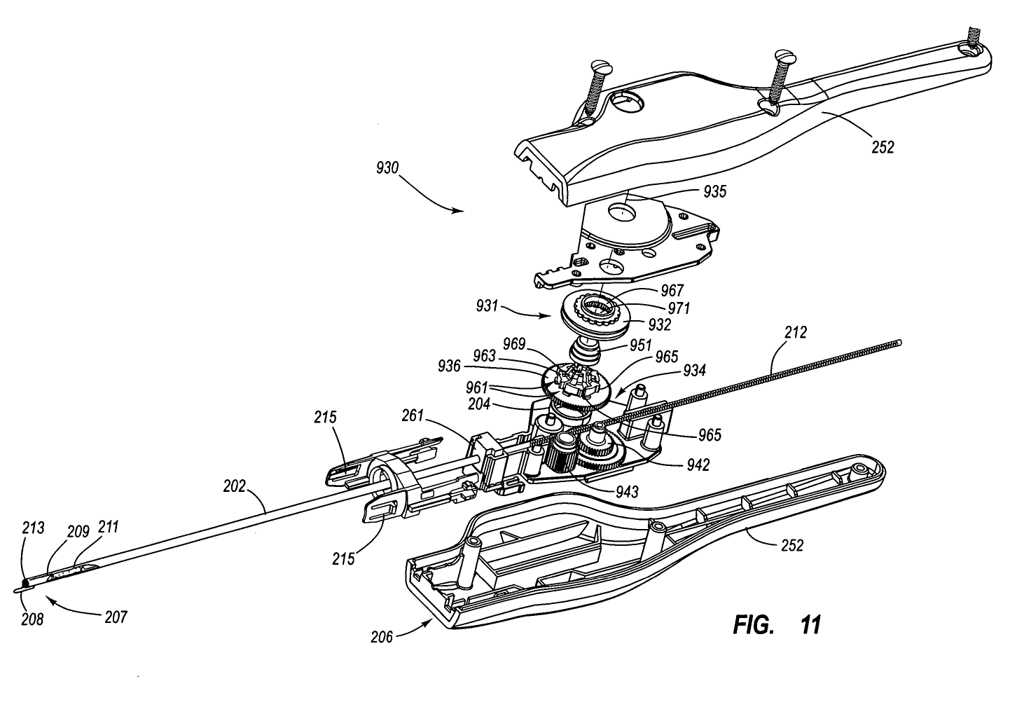

Referring next to FIG. 11, another embodiment of a selectably disengagable automatic driving mechanism 930 is shown. The selectably disengagable automatic driving mechanism 930 of FIG. 11 may replace the selectably disengagable gearbox assembly 629 shown in FIG. 8 within the closure device 200 (FIG. 7A). Similar to the embodiment of FIG. 8, the selectably disengagable automatic driving mechanism 930 of FIG. 11 includes the suture 204 at least partially wound about a spool 932 of a first gear and spool assembly 931. The first gear and spool assembly 931 includes both the spool 932 and a first gear 936 arranged on a first axis 935. However, according to the embodiment of FIG. 11, the first gear 936 and the spool 932 form a manually operated clutch therebetween. The clutch may be used to selectively connect and disconnect the first gear 936 from the spool 932. The clutch comprises a plurality of release fingers 961 in FIG. 11. The release fingers 961 are arranged substantially in a circle. A first component 963 of the release fingers 961 is cantilevered from the first gear 936 and extends normal to the first gear 936. A protrusion 965 of the first component 963 extends radially outward and is received by a mating recess 967 of the spool 932. A second component 969 of the release fingers 961 arcs substantially normal to the first component 963 and the first gear 936. The second component 969 of each of the release fingers 961 extends through a central hole 971 of the spool 932. An actuator button 951 fits over and contacts the second components 969 of each of the release fingers 961.

The fit of the protrusions 965 of the first gear 936 with the mating recesses 967 of the spool 932 causes the first gear 936 and spool 932 to rotate together at an identical angular velocity. However, when the actuator button 951 is depressed, the actuator button 951 slides along the arcs of the second component 969, forcing each of the release fingers 961 radially inward. The radial inward displacement of the release fingers 961 at least partially removes the protrusions 965 from the mating recesses 967, allowing independent rotation of the spool 932 with respect to the first gear 936. Therefore, similar to the arrangement described above with reference to FIGS. 7A-10, after the incision track plug 211 and thus the sealing plug 210 are driven toward the anchor 208, the selectably disengagable automatic driving mechanism 930 is disengaged or disabled, allowing the suture 204 to safely unwind without further tamping. The suture 204 is then exposed to the operator for convenient cutting.

The remaining components of the selectably disengagable automatic driving mechanism 930 may be similar to the embodiment of FIG. 8. Transducing the torsional motive force provided by the spool 932 to the linear tamping force is achieved by a gear train 934. The gear train 934 may include the first gear 936 and second and third gears 942, 943. As shown, the second gear 942 engages and drives the third gear 943, and the third gear 943 drives a tamping tube 212 or other sealing plug driving device. The second gear 942 of FIG. 11 does not, however, include an annular groove interlocking with the tamping tube 212.

The preceding description has been presented only to illustrate and describe expemplary embodiments of inveniton. It is not intended to be exhaustive or to limit the invention to any precise form disclosed. Many modifications and variations are possible in light of the above teaching. It is intended that the scope of the invention be defined by the following claims.

What is claimed is:

1. A tissue puncture closure device for partial insertion into and sealing of an internal tissue wall puncture, comprising:
a filament extending from a first portion of the closure device to a second portion of the closure device;
an anchor for insertion through the internal tissue wall puncture attached to the filament at the second portion of the closure device;
a sealing plug slidingly attached to the filament adjacent to the anchor;
an incision track plug slidingly attached to the filament adjacent to the sealing plug.

2. A tissue puncture closure device for partial insertion into and sealing of an internal tissue wall puncture according to claim 1 wherein the incision track plug comprises a rigid biologically resorbable core with an adsorbent biologically resorbable outer lining.

3. A tissue puncture closure device for partial insertion into and sealing of an internal tissue wall puncture according to claim 1 wherein the incision track plug comprises a generally cylindrical hollow core covered with collagen.

4. A tissue puncture closure device for partial insertion into and sealing of an internal tissue wall puncture according to claim 1 wherein the incision track plug comprises a generally cylindrical biologically resorbable polymer covered by a collagen pad.

5. A tissue puncture closure device for partial insertion into and sealing of an internal tissue wall puncture according to claim 1 wherein the incision track plug is adapted to remain in an incision track and adsorb blood from surrounding skin tissue.

6. A tissue puncture closure device for partial insertion into and sealing of an internal tissue wall puncture according to claim 1, further comprising a tamping tube slidingly disposed about the filament proximal of the incision track plug.

7. A tissue puncture closure device for partial insertion into and sealing of an internal tissue wall puncture according to claim 1, further comprising a tamping tube having an inner diameter, the tamping tube slidingly disposed about the filament proximal of the incision track plug;
wherein the incision track plug comprises a core having an outer diameter larger than the inner diameter of the tamping tube.

8. A tissue puncture closure device for partial insertion into and sealing of an internal tissue wall puncture according to claim 1, further comprising a tamping tube that, when forced distally, presses the incision track plug toward the sealing plug, and the incision track plug compresses the sealing plug toward the anchor.

9. A tissue closure device for partial insertion into and sealing of an internal tissue wall puncture according to claim 1, further comprising a selectably disengagable automatic driving mechanism for automatically tamping or cinching the incision track plug and the sealing plug toward the second end upon withdrawal of the closure device from the internal puncture.

10. A tissue puncture closure device for partial insertion into and sealing of and internal tissue wall puncture according to claim 1, further comprising a tamping tube disposed adjacent to the incision track plug;
wherein the tamping tube is driven by a selectably disengagable automatic driving mechanism to force the incision track plug distally and tamp the sealing plug.

11. A tissue puncture closure device for partial insertion into and sealing of an internal tissue wall puncture according to claim 1, further comprising:
a selectably disengagable automatic driving mechanism operatively connected to the incision track plug, the selectably disengagable automatic driving mechanism comprising:
a first gear and a spool assembly arranged on a first axis with a portion of the filament wound thereon;
a manually operated clutch between the first gear and the spool assembly;
wherein the clutch operably connects and disconnects the spool to the first gear.

12. A tissue puncture closure device for partial insertion into and sealing of an internal tissue wall puncture according to claim 11, further comprising:
a second gear on a second axis adjacent to the first gear;
a third gear on a third axis adjacent to the second gear.

13. A tissue puncture closure device for partial insertion into and sealing of a tissue puncture in an internal tissue wall accessible through a percutaneous incision track, comprising:
an anchor for disposition on a distal side of the internal tissue wall;
a sealing plug for disposition on a proximal side of the internal tissue wall;
an incision track plug for disposition in the percutaneous incision track proximal of the sealing plug;
a connector attached to and anchored at a distal end to the anchor, wherein the sealing plug and the incision track plug are slidably attached to the connector proximal of the anchor;
a tamping device disposed on the connector for driving the incision track plug and the sealing plug along the connector distally towards the anchor.

14. A tissue puncture closure device for partial insertion into and sealing of a tissue puncture in an internal tissue wall accessible through a percutaneous incision according to claim 13 wherein the incision track plug comprises a stiff biologically resorbable core with an adsorbent biologically resorbable outer lining.

15. A tissue puncture closure device for partial insertion into and sealing of a tissue puncture in an internal tissue wall accessible through a percutaneous incision according to claim 13 wherein the incision track plug comprises a generally cylindrical hollow core covered with collagen, wherein the connector is threaded through the cylindrical hollow core.

16. A tissue puncture closure device for partial insertion into and sealing of a tissue puncture in an internal tissue wall accessible through a percutaneous incision according to claim 13 wherein the incision track plug is adapted to compress the sealing plug toward the anchor, remain in an incision track, and adsorb blood from surrounding tissue.

17. A tissue puncture closure device for partial insertion into and sealing of a tissue puncture in an internal tissue wall accessible through a percutaneous incision according to claim 13 wherein the incision track plug comprises a biologically resorbable member approximately one inch long.

18. A method of sealing a tissue puncture in an internal tissue wall accessible through a percutaneous incision, comprising:
setting an anchor inside the internal tissue wall through the puncture;
deploying a sealing plug and an incision track plug in the percutaneous incision;
tamping the incision track plug and the sealing plug toward the anchor;
seating the sealing plug against the puncture.

19. A method of sealing a tissue puncture in an internal tissue wall accessible through a percutaneous incision according to claim 18, further comprising adsorbing blood from surrounding tissue of the percutaneous incision with the incision track plug.

20. A method of sealing a tissue puncture in an internal tissue wall accessible through a percutaneous incision according to claim 18, further comprising leaving the anchor, sealing plug, and incision track plug in a patient body.

21. A method of sealing a tissue puncture in an internal tissue wall accessible through a percutaneous incision according to claim 18 wherein the tamping comprises manually tamping the incision track plug with a tamping tube, wherein the incision track plug tamps the sealing plug.

22. A method of sealing a tissue puncture in an internal tissue wall accessible through a percutaneous incision according to claim 18 wherein the tamping comprises:
withdrawing a closure device carrying the sealing plug and the incision track plug from the tissue puncture;
automatically transducing a motive force generated by withdrawal of the closure device in a first direction to a cinching or tamping force in a second direction.

23. A method of sealing a tissue puncture in an internal tissue wall accessible through a percutaneous incision according to claim 18 wherein the tamping comprises:
withdrawing a closure device carrying the sealing plug and the incision track plug from the tissue puncture;
automatically transducing a motive force generated by withdrawal of the closure device in a first direction to a cinching or tamping force in a second direction;
and further comprising manually disabling the tamping force in the second direction.

24. A method of sealing a tissue puncture in an internal tissue wall accessible through a percutaneous incision according to claim 18 wherein seating the sealing plug comprises cinching the sealing plug and the anchor together across the puncture.

25. A method of sealing a tissue puncture in an internal tissue wall accessible through a percutaneous incision according to claim 18 wherein the incision track plug comprises a stiff biologically resorbable core with an adsorbent biologically resorbable outer lining.

26. A method of sealing a tissue puncture in an internal tissue wall accessible through a percutaneous incision, comprising:
providing a tissue puncture closure device comprising a filament connected at its distal end to an anchor and to a sealing plug and incision track plug located proximal of the anchor for disposition and anchoring about the tissue puncture;
inserting the tissue puncture closure device into the percutaneous incision;
deploying the anchor into the tissue puncture;
at least partially withdrawing the closure device from the percutaneous incision;
tamping the incision track plug and sealing plug toward the anchor upon withdrawal of the closure device from the internal tissue wall puncture.

27. A method of sealing a tissue puncture in an internal tissue wall accessible through a percutaneous incision according to claim 26 wherein the tissue puncture closure device comprises an automatic tamping device; the method further comprising:
disengaging the automatic tamping device;
retracting the tissue puncture closure device;
exposing the filament;
cutting the filament;
leaving the anchor and the sealing plug at the tissue puncture and the incision track plug in the percutaneous incision.
